(19) United States
(12) Patent Application Publication
(10) Pub. No.: US 2009/0142291 A1
(43) Pub. Date: Jun. 4, 2009

(54) PIGMENTED COSMETIC AND PERSONAL CARE COMPOSITION AND PROCESS FOR PREPARING THE SAME

(75) Inventor: Robert G. DROMAN, Akron, OH (US)

Correspondence Address: CURATOLO SIDOTI CO., LPA, 24500 CENTER RIDGE ROAD, SUITE 280, CLEVELAND, OH, 44145, US

(73) Assignee: Gayson Silicone Dispersments, Inc., Barberton, OH (US)

(21) Appl. No.: 12/245,273

(22) Filed: Oct. 3, 2008

Related U.S. Application Data
(60) Provisional application No. 60/977,915, filed on Oct. 5, 2007.
(60) Provisional application No. 61/052,670, filed on May 13, 2008.

(51) Int. Cl. A61K 31/74

(52) U.S. Cl. 424/78.02

(57) ABSTRACT

A pigment dispersed in a silicone polymer is combined with at least one cosmetic component or at least one dematologically acceptable and active agent to prepare a wide variety of cosmetic and personal care compositions. The pigment may be dispersed in a polysiloxane, such as an alkylpolysiloxane, to provide a pigment dispersion that is easily blended with cosmetically acceptable components or dematologically active agents to provide a pigmented cosmetic or personal care composition.

### CROSS REFERENCE TO RELATED APPLICATIONS

The present application claims the benefit of the filing date, under 35 U.S.C. §119(e), from U.S. Provisional Application Ser. No. 60/977,915, filed Oct. 5, 2007 and from U.S. Provisional Application Ser. No. 61/052,670, filed May 13, 2008.

### TECHNICAL FIELD

Disclosed are cosmetic and personal care compositions and a process for preparing the same. More particularly, disclosed pigmented cosmetic and personal care compositions and a process for preparing pigmented cosmetic and personal care compositions using a pigment dispersion to impart color to the compositions.

### BACKGROUND

A pigment is a substance that imparts color to another substance or material to which it is added. Pigments impart color to other substances through the selective absorption and reflection of certain wavelengths of light. Pigments are typically used for coloring, for example, cosmetics, fabrics, inks, paints, and plastics. Many pigments, such as solid inorganic pigments, are generally insoluble in most liquid vehicles, such as organic solvents and water.

It is desirable to impart color to cosmetics. Traditionally, dry powdered pigments are introduced into a cosmetically acceptable medium. However, the use of powdered pigments is messy and presents a risk of contaminating other products in a cosmetic laboratory or production facility. Furthermore, the use of airborne dry pigment powders also exposes the cosmetic formulators to health risks associated with inhalation of the dry powdered pigment.

It is widely known that it is difficult to uniformly disperse powdered pigments into cosmetic compositions. When formulating a cosmetic composition, the use of dry powdered pigments also requires a substantial amount time in order to ensure that the pigment is uniformly dispersed throughout the cosmetic composition so as to avoid inconsistent or streaking color.

Therefore, it is desirable in the art to provide a cosmetic composition that includes a cosmetically acceptable pigment and a process for easily and efficiently imparting the pigment to the cosmetic composition.

### SUMMARY

Disclosed is a colored cosmetic composition comprising a mixture of at least one cosmetically acceptable component and a pigment dispersion. According to certain embodiments, the pigment dispersion comprises a color deviation expressed as a delta-E value of 1.5 or less from a previously established color standard.

According to certain illustrative embodiments, the colored cosmetic composition comprises a mixture of at least one cosmetically acceptable component and a silicone pigment dispersion.

According to other illustrative embodiments, the colored cosmetic composition comprises a mixture of at least one cosmetically acceptable component and a thixotropic pigment dispersion.

According to further illustrative embodiments, the colored cosmetic composition comprises a mixture of at least one cosmetically acceptable component and a thixotropic silicone pigment dispersion.

Also disclosed is a process for preparing a colored cosmetic composition comprised of mixing together at least one cosmetically acceptable component and a pigment dispersion. According to certain embodiments, the process comprises combining a pigment dispersion comprising a color deviation expressed as a delta-E value of 1.5 or less from a previously established color standard with at least one cosmetically acceptable component.

According to certain illustrative embodiments, the process for preparing a pigmented cosmetic composition comprises mixing together at least one cosmetically acceptable component and a silicone pigment dispersion.

According to other illustrative embodiments, the process for preparing a colored cosmetic composition comprises mixing together at least one cosmetically acceptable component and a thixotropic pigment dispersion.

According to further illustrative embodiments, the process for preparing a colored cosmetic composition comprises mixing together at least one cosmetically acceptable component and a thixotropic silicone pigment dispersion.

Also disclosed is a colored personal care composition comprising a mixture of at least one dermatologically active agent and a pigment dispersion.

Also disclosed is a personal care composition comprising a mixture of at least one dermatologically active agent and a pigment dispersion. According to certain embodiments, the pigment dispersion comprises color deviation expressed as a delta-E value of 1.5 or less from a previously established color standard.

According to certain illustrative embodiments, the colored personal care composition comprises a mixture of at least one dermatologically active agent and a silicone pigment dispersion.

According to other illustrative embodiments, the colored personal care composition comprises a mixture of at least one dermatologically active agent and a thixotropic pigment dispersion.

According to further illustrative embodiments, the colored personal care composition comprises a mixture of at least one dermatologically active agent and a thixotropic silicone pigment dispersion.

Also disclosed is a process for preparing a colored personal care composition comprised of mixing together at least one dermatologically active agent and a pigment dispersion.

Also disclosed is a process for preparing a colored personal care composition comprised of mixing together at least one dermatologically active agent and a pigment dispersion. According to certain embodiments, the process comprises combining a pigment dispersion comprising a color deviation expressed as a delta-E value of 1.5 or less from a previously established color standard with at least one dermatologically active agent.

According to certain illustrative embodiments, the process for preparing a colored personal care composition comprises mixing together at least one dermatologically active agent and a silicone pigment dispersion.

According to other illustrative embodiments, the process for preparing a colored personal composition comprises mixing together at least one dermatologically active agent and a thixotropic pigment dispersion.

According to further illustrative embodiments, the process for preparing a colored personal care composition comprises mixing together at least one dermatologically active agent and a thixotropic silicone pigment dispersion.

### DETAILED DESCRIPTION

A cosmetic composition comprising a blend or mixture of at least one cosmetically acceptable component and at least one cosmetically or dermatologically acceptable pigment. The cosmetically or dermatologically acceptable pigment may be introduced into the cosmetic component in the form of a thixotropic dispersion. As used herein, the term “thixotropic” refers to a pigment dispersion having a certain viscosity in the absence of the application of force, which becomes less viscous in response to the application of force, and then become more viscous when the applied force is removed. The use of the silicone pigment dispersion, such as a thixotropic silicone pigment dispersion, avoids the step of having to add powdered pigments directly to a cosmetically acceptable component and promotes the homogenous or uniform dispersion of the pigment throughout the cosmetic composition.

The pigment to be added to the cosmetically acceptable component to prepare the colored cosmetic composition is dispersed within a silicone polymer. When used herein, the term “the pigment” refers to a single species of pigment or a mixture of two or more different species of pigments. Therefore, it should be noted that any given cosmetic composition may include more than one different species of pigment.

According to certain embodiments, the pigmented cosmetic composition may be prepared by blending together at least one cosmetic component and a silicone pigment dispersion including 1 to 10 different species of pigment.

According to certain embodiments, the pigmented cosmetic composition may be prepared by blending together at least one cosmetic component and a thixptropic silicone pigment dispersion including 1 to 10 different species of pigment.

According to alternative embodiments, the pigmented cosmetic composition may be prepared by blending together at least one cosmetic component and a silicone pigment dispersion including 3 to 10 different species of pigment.

According to alternative embodiments, the pigmented cosmetic composition may be prepared by blending together at least one cosmetic component and a thixotropic silicone pigment dispersion including 3 to 10 different species of pigment.

According to alternative embodiments, the pigmented cosmetic composition may be prepared by blending together at least one cosmetic component and a silicone pigment dispersion including 3 to 4 different species of pigment.

According to alternative embodiments, the pigmented cosmetic composition may be prepared by blending together at least one cosmetic component and a thixotropic silicone pigment dispersion including 3 to 4 different species of pigment.

It should be noted that more than one silicone dispersion, each having one or more pigment dispersed therein, may be added to the cosmetic component to prepare a colored cosmetic composition.

According to certain illustrative embodiments, the pigment is dispersed within a silicone polymer, such as a polysiloxane. Without limitation, suitable polysiloxanes include organopolysiloxanes. By way of illustration, the organopolysiloxane may comprise the following chemical structure:

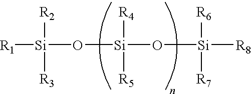

wherein R₁-R₇ are each independently selected from H, —CH₃, —CH₂CH₃.OH, Vinyl, and any other chemical group or combinations of different chemical groups which would provide a cosmetically or dermatologically acceptable silicone polymer, such as those silicone polymers listed in the International Cosmetic and Ingredient Dictionary and Handbook;
and wherein n is an integer from about 3 to about 1,000,000.

Without limitation, the initial viscosity of an illustrative silicone polymer is in the range of about 1 to about 1 billion centistokes (cSt). According to certain embodiments, the initial viscosity of a useful silicone polymer is about 350,000 cSt. The pigment or mixture of more than one pigment may also be dispersed in a high molecular weight silicone gum. Without limitation, a suitable high molecular weight silicone gum may have a Williams Plasticity of about 100 to about 150, or about 100 to about 130. According to certain embodiments, the pigment or mixture of more than one pigment may be dispersed in a high molecular weight silicone gum having a Williams Plasticity of about 117.

According to certain embodiments, the pigment may be dispersed in a dimethylpolysiloxane of the following structure:

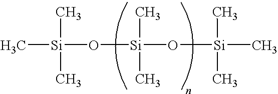

Dimethylpolysiloxane is also referred to in the International Cosmetic and Ingredient Dictionary and Handbook as “dimethicone”. It comprises a mixture of fully methylated siloxane polymers end capped with various chemical groups. The dimethylpolysiloxane may be end capped with the same or different organic groups. Without limitation, and only by way of exemplification, the dimethylpolysiloxane may be end capped with trialkylsiloxy groups. For example, the dimethylsiloxane groups may be end capped with a trimethylsiloxy group, a triethylsiloxy group or combinations thereof.

According to certain embodiments, the pigment dispersion comprises from about 10 to about 99.99 weight percent silicone polymer, from about 0.01 to about 80 weight percent pigment and from about 0.01 to about 50 weight percent rheology modifier. Without limitation, a suitable formulation of the pigment dispersion comprises from about 24 to about 90 weight percent silicone polymer, from about 10 to about 76 weight percent pigment and from about 1 to about 10 weight percent rheology modifier. An additionally suitable formulation of the pigment dispersion comprises from about 60 to about 80 weight percent silicone polymer, from about 10 to about 50 weight percent pigment and from about 1 to about 10 weight percent rheology modifier. An useful pigment dispersion for use in preparing the cosmetic composition may include about 55 weight percent silicone polymer, about 43 weight percent pigment and about 2 weight percent rheology modifier.

According to certain embodiments, the solid pigment combined with the silicone polymer may be mechanically manipulated, for example, by grinding, to reduce at least a portion of the solid pigment particles substantially to their primary particle size. A rheology modifier may be added, and the various combinations of the silicone polymer, pigment and rheology modifier, are subjected to high shearing mixing to uniformly disperse the solid pigment throughout the silicone polymer. If the particles of a given pigment included in the silicone dispersion are of significantly differing sizes, the resulting color tends to be more random and inconsistent. On the contrary, the use of a silicone pigment dispersion wherein the pigment particles have been reduced substantially to their primary particle sizes contributes to a dispersion of stable and uniform color. For silicone pigment dispersions containing a combination of more than one species of pigment, it should be noted that the primary particle sizes for the different species of pigment may also be different. The cosmetic composition and process of preparation of the cosmetic composition encompasses the use of silicone pigment dispersions which include combinations of more than one type of pigment species having different primary particle sizes.

The pigments which may be dispersed in the silicone polymer and used to prepare colored cosmetic compositions include any known natural or synthetic pigment that is generally acceptable for use in formulating cosmetic compositions. These pigments include inorganic pigments, organic pigments or mixtures of inorganic and organic pigments.

Without limitation, suitable examples of inorganic pigments include red iron oxide, yellow iron oxide, black iron oxide, brown iron oxide, titanium dioxide, zinc oxide, zirconium oxide, cerium oxide, chromium oxide, chromium hydroxide, chromium hydrate, manganese violet, ferric ferrocyanide, guanine (natural pearl) mica, sericite, talc, magnesium carbonate, calcium carbonate, alumina hydroxide, barium sulfate, beta-carotene, annatto, chlorophyllin-copper complex, lycopene, henna, bronze powder, (metallic), copper powder (metallic), silver powder (metallic), gold powder (metallic), ultramarine blues, Lake pigments and the like.

According to certain illustrative embodiments, the pigments that may be incorporated into the dispersion include the iron oxide pigments, such as red iron oxide, yellow iron oxide, black iron oxide, brown iron oxide, titanium dioxide and mixtures thereof.

Without limitation, suitable examples of organic pigments include those which are FD&C Blue #1, FD&C Yellow #5, FD&C Yellow #6, FD&C Yellow #7, FD&C Yellow #8, FD&C Yellow #10, FD&C Red #2, FD&C Red #4, FD&C Red #6, FD&C Red #7, FD&C Red #9, FD&C Red #17, FD&C Red #19, FD&C Red #21, FD&C Red #22, FD&C Red #27, FD&C Red #28, FD&C Red #30, FD&C Red #31, FD&C Red #33, FD&C Red 34, FD&C Red #36, FD&C Red #40, D&C Violet #2, Ext D&C Violet #2, FD&C Green #3, D&C Green #5, D&C Green #6, D&C Green #8, D&C Orange #4, D&C Orange #5, D&C Brown #1, D&C Black #2, carmine and mixtures thereof.

The pigment dispersion also includes a rheology modifier. The rheology modifier is included in the pigment dispersion in an amount effective to provide a stable pigment dispersion having a controllable viscosity, thereby making it easy to uniformly introduce pigment into a cosmetically acceptable component or medium. A suitable rheology modifier that may be incorporated into the pigment dispersion is fumed silica. According to certain illustrative embodiments, the fumed silica may be chemically altered or modified by the addition of different chemical groups to the surface of the silica. A suitable chemically modified fumed silica for use in the pigment dispersion comprises silica dimethyl silylate. Silica dimethyl silylate is a fumed silica derivative where some of the hydroxyl groups on the surface of the fumed silica have been replaced with, but not limited to, dimethyl silyl groups.

According to certain illustrative embodiments, the pigment dispersion used to prepare the colored cosmetic composition exhibits a delta-E (dE) value of about 1 or less. According to certain embodiments, the pigment dispersion used to prepare the colored cosmetic composition exhibits a delta-E (dE) value of 1.5 or less. According to further embodiments, the pigment dispersion used to prepare the colored cosmetic composition exhibits a Delta-E (dE) value of 1.0 or less.

The Delta-E value is a number that represents the “distance” or deviation between two colors. Generally speaking, a Delta-E value of 1.0 represents the smallest color difference between two colors that is detectable by the unassisted human eye, and any value less than 1.0 is virtually imperceptible by the human eye. Delta-E of the colored cosmetic composition may be determined by, for example, the procedure set forth in ASTM Standard D2244-07. ASTM D2244-07 covers the determination of color differences (magnitude and direction) and tolerances between colored specimens that are obtained from instrumentally measured color coordinates based on daylight illumination. Color difference may be calculated from the measured color data using equations CMC, DIN99, CIE dE76, CIE dE94, CIE dE2000, or similar industry-accepted mathematical equations. A custom color pigment dispersion exhibiting a delta-E value of 1.5 or less, or of 1.0 or less, signifies a pigment dispersion having a tight color tolerance from an established color standard.

Additionally disclosed is a method for providing to a cosmetic producer a custom formulated pigment dispersion for preparing a colored cosmetic composition. The method includes accepting a request from a cosmetic producer to prepare a pigment dispersion for use in the preparation of a colored cosmetic composition, the pigment dispersion having a desired color standard established by the cosmetic producer, determining the type and amount of raw materials that are needed to attain the desired color standard, and formulating the pigment dispersion with the type and amount of raw materials to attain to desired color standard.

According to certain illustrative embodiments, the method for providing a custom formulated pigment dispersion to a cosmetic producer includes accepting a request from a cosmetic producer to prepare a custom formulated pigment dispersion having a desired color established by the cosmetic producer, determining the type and amount of raw materials that are needed to attain the desired color and formulating the pigment dispersion with the type and amount of raw materials to attain to desired color, wherein the custom formulated pigment dispersion exhibits a color deviation as expressed by a delta-E value of 1.5 or less from the cosmetic producer established color standard.

According to certain illustrative embodiments, the method for providing a custom formulated pigment dispersion to a cosmetic producer includes accepting a request from a cosmetic producer to prepare a custom formulated pigment dispersion having a desired color established by the cosmetic producer, determining the type and amount of raw materials that are needed to attain the desired color and formulating the pigment dispersion with the type and amount of raw materials to attain to desired color, wherein the custom formulated pigment dispersion exhibits a color deviation as expressed by a delta-E value of 1.0 or less from the cosmetic producer established color standard.

According to further illustrative embodiments, method for providing a custom formulated pigment dispersion to a cosmetic producer includes accepting a request from a cosmetic producer to prepare a pigment dispersion having a desired color, determining the type and amount of raw materials that are needed to attain the desired color and formulating the pigment dispersion with the type and amount of raw materials to attain to desired color, wherein the type of raw materials include at least one colorant and at least one carrier for the at least one colorant.

According to yet further illustrative embodiments, method for providing a custom formulated pigment dispersion to a cosmetic producer includes accepting a request from a party to prepare a pigment dispersion having a desired color, determining the type and amount of raw materials that are needed to attain the desired color and formulating the pigment dispersion with the type and amount of raw materials to attain to desired color, wherein the type of raw materials include at least one colorant comprises at least one pigment and wherein the carrier comprises a silicone polymer. The at least one pigment and silicone polymer are blended together to provide a silicone pigment dispersion, and the silicone pigment dispersion is then blended with the at least one cosmetically acceptable component to provide a custom formulated colored cosmetic composition.

The pigment dispersion may be used to formulate a wide variety of cosmetics. As used herein, the term “cosmetic” refers to any substance that is rubbed, poured, sprinkled, sprayed on, introduced into, or otherwise applied to the human body or any part thereof for the purpose of beautifying, cleansing, altering, enhancing or promoting the attractiveness of the skin, or protecting the skin. A subset of cosmetics is called “make-up,” which refers primarily to colored products intended to alter the appearance of the user's face and neck areas.

The pigment dispersion may be used to prepare a wide variety of pigmented cosmetic compositions. Without limitation, the pigment dispersions may be used to prepare base, concealers, foundations in liquid, cream, mousse or powder forms, bronzers, face powders, face illuminators, lipstick, lip gloss, lip liner, lip plumper, lip luster, lip conditioners, lip boosters, rouge, blush (also referred to as blushers), mascara, lash extenders, lash conditioners, eyebrow liners, eye shadow, eye shimmer, eye glitter, eye pencils, eyebrow creams, eyebrow waxes, eyebrow gels, and eyebrow powders, nail polish to color fingernails and toenails.

The pigment dispersions may be used to prepare a wide variety of pigmented skin care products. Without limitation, the pigment dispersions may be used to formulate face and body creams, lotions, moisturizers, ointments, suntan oils, suntan lotions, suntan sprays, sun blocks, and sun screens.

The pigment dispersions may also be used to formulation bath soaps, bath and shower gels, bath oils, bath salts, bubble baths, bath butters, hair shampoos, hair conditioners, hair gels, hair mousses, and hair colorants.

The use of the pigment dispersion permits the pigment to be easily and quickly introduced into a cosmetic composition during the formulation of the cosmetic composition and results in more uniform dispersion of the pigment throughout the cosmetic composition, while minimizing settling or separating out of the cosmetic composition.

While the pigmented cosmetic composition and process for preparing the same have been described in connection with various illustrative embodiments, it is to be understood that other similar embodiments may be used or modifications and additions may be made to the described embodiments for performing the same function disclosed herein without deviating therefrom. The embodiments described above are not necessarily in the alternative, as various embodiments may be combined to provide the desired characteristics. Therefore, the pigmented cosmetic composition and process for preparing the same should not be limited to any single embodiment, but rather construed in breadth and scope in accordance with the recitation of the appended claims.

1. A colored cosmetic composition comprising at least one cosmetically acceptable component and a silicone pigment dispersion.

2. The colored cosmetic composition of claim 1, wherein said silicone pigment dispersion exhibits a color deviation expressed by a delta-E value of 1.5 or less from a previously established color standard.

3. The cosmetic composition of claim 2, wherein said silicone polymer comprises a polysiloxane.

4. The cosmetic composition of claim 3, wherein said polysiloxane comprises a polyalkylsiloxane.

5. The cosmetic composition of claim 4, wherein said polyalkylsiloxane comprises the following chemical structure: 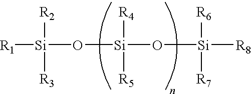
wherein R₁-R₇ are each independently selected from H, —OH, —CH₃, —CH₂CH₃, vinyl and combinations thereof; and
wherein n is an integer from about 3 to about 1,000,000.

6. The cosmetic composition of claim 5, wherein said polyalkylsiloxane comprises dimethylpolysiloxane.

7. The cosmetic composition of claim 6, wherein said pigment comprises an inorganic pigment, an organic pigment, or mixtures thereof.

8. The cosmetic composition of claim 7, wherein said pigment comprises an inorganic pigment.

9. The cosmetic composition of claim 8, wherein said inorganic pigment is selected from the group consisting of red iron oxide, yellow iron oxide, black iron oxide, brown iron oxide, titanium dioxide, zinc oxide, zirconium oxide, cerium oxide, chromium oxide, chromium hydroxide, chromium hydrate, manganese violet, ferric ferrocyanide, guanine (natural pearl) mica, sericite, talc, magnesium carbonate, calcium carbonate, alumina hydroxide, barium sulfate, beta-carotene, annatto, chlorophyllin-copper complex, lycopene, henna, bronze powder, (metallic), copper powder (metallic), silver powder (metallic), gold powder (metallic), ultramarine blues and mixtures thereof.

10. The cosmetic composition of claim 7, wherein said pigment comprises an organic pigment.

11. The cosmetic composition of claim 10, wherein said organic pigment is selected from the group consisting of FD&C Blue#1, FD&C Yellow#5, FD&C Yellow#6, FD&C Yellow#7, FD&C Yellow#8, FD&C Yellow#10, FD&C Red#2, FD&C Red #4, FD&C Red#6, FD&C Red#7, FD&C Red #9, FD&C Red #17, FD&C Red #19, FD&C Red 21, FD&C Red #22, FD&C Red #27, FD&C Red #28, FD&C Red #30, FD&C Red #31, FD&C Red #33, FD&C Red 34, FD&C Red #36, FD&C Red #40, D&C Violet#2, Ext D&C Violet#2, FD&C Green#3, D&C Green#5, D&C Green#6, D&C Green#8, D&C Orange#4, D&C Orange #5, D&C Brown#1, D&C Black#2, carmine, and mixtures thereof.

12. The cosmetic composition of claim 2, wherein said pigment dispersion further comprises a rheology modifier.

13. The cosmetic composition of claim 12, wherein said rheology modifier comprises a fumed silica.

14. The cosmetic composition of claim 15, wherein the surface of said fumed silica is chemically modified.

15. The cosmetic composition of claim 14, wherein said modified fumed silica comprises silica dimethyl silylate.

16. A process for preparing a colored cosmetic composition comprising mixing a silicone pigment dispersion with at least one cosmetically acceptable component.

17. A process for preparing a colored cosmetic composition of claim 16, wherein said silicone pigment dispersion exhibits a color deviation expressed by a delta-E value of 1.5 or less from a previously established color standard.

18. The process of claim 17, wherein said silicone polymer comprises a polysiloxane.

19. The process of claim 18, wherein said polysiloxane comprises a polyalkylsiloxane.

20. The process of claim 19, wherein said polyalkylsiloxane comprises the following chemical structure: 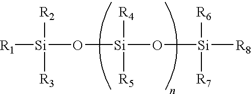
wherein R₁-R₇ are each independently selected from
H, OH, CH₃, CH₂CH₃, vinyl and combinations thereof; and
wherein n is an integer from about 3 to about 1,000,000.

21. The process of claim 20, wherein said polyalkylsiloxane comprises dimethylpolysiloxane.

22. The process of claim 17, wherein said pigment comprises an inorganic pigment, an organic pigment, or mixtures thereof.

23. The process of claim 22, wherein said pigment comprises an inorganic pigment.

24. The process of claim 23, wherein said inorganic pigment is selected from the group consisting of red iron oxide, yellow iron oxide, black iron oxide, brown iron oxide, titanium dioxide, zinc oxide, zirconium oxide, cerium oxide, chromium oxide, chromium hydroxide, chromium hydrate, manganese violet, ferric ferrocyanide, guanine (natural pearl) mica, sericite, talc, magnesium carbonate, calcium carbonate, alumina hydroxide, barium sulfate, beta-carotene, annatto, chlorophyllin-copper complex, lycopene, henna, bronze powder, (metallic), copper powder (metallic), silver powder (metallic), gold powder (metallic), ultramarine blues and mixtures thereof.

25. The process of claim 24, wherein said pigment comprises an organic pigment.

26. The process of claim 25, wherein said organic pigment is selected from the group consisting of FD&C Blue#1, FD&C Yellow#5, FD&C Yellow#6, FD&C Yellow#7, FD&C Yellow#8, FD&C Yellow#10, FD&C Red#2, FD&C Red #4, FD&C Red#6, FD&C Red#7, FD&C Red #9, FD&C Red #17, FD&C Red #19, FD&C Red 21, FD&C Red #22, FD&C Red #27, FD&C Red #28, FD&C Red #30, FD&C Red #31, FD&C Red #33, FD&C Red 34, FD&C Red #36, FD&C Red #40, D&C Violet#2, Ext D&C Violet#2, FD&C Green#3, D&C Green#5, D&C Green#6, D&C Green#8, D&C Orange#4, D&C Orange #5, D&C Brown#1, D&C Black#2, carmine, and mixtures thereof.

27. The process of claim 17, wherein said pigment dispersion further comprises a rheology modifier.

28. The process of claim 27, wherein said rheology modifier comprises a fumed silica.

29. The process of claim 28, wherein the surface of said fumed silica is chemically modified.

30. The process of claim 29, wherein said modified fumed silica comprises silica dimethyl silylate.

31. A colored personal care composition comprising at least one dematologically active agent and a silicone pigment dispersion.

32. The colored personal care composition of claim 31, wherein said silicone pigment dispersion exhibits a color deviation from a previously established color standard expressed by a delta-E value of 1.5 or less.

33. The colored personal care composition of claim 32, wherein said silicone polymer comprises a polysiloxane.

34. The colored personal care composition of claim 33, wherein said polysiloxane comprises a polyalkylsiloxane.

35. The colored personal care composition of claim 34, wherein said polyalkylsiloxane comprises the following chemical structure: 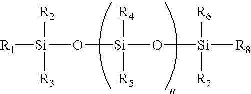
wherein R₁-R₇ are each independently selected from H, —OH, —CH₃, —CH₂CH₃, vinyl and combinations thereof; and
wherein n is an integer from about 3 to about 1,000,000.

36. The colored personal care composition of claim 35, wherein said polyalkylsiloxane comprises dimethylpolysiloxane.

37. The colored personal care composition of claim 36, wherein said pigment comprises an inorganic pigment, an organic pigment, or mixtures thereof.

38. The colored personal care composition of claim 37, wherein said pigment comprises an inorganic pigment.

39. The colored personal care composition of claim 38, wherein said inorganic pigment is selected from the group consisting of red iron oxide, yellow iron oxide, black iron oxide, brown iron oxide, titanium dioxide, zinc oxide, zirconium oxide, cerium oxide, chromium oxide, chromium hydroxide, chromium hydrate, manganese violet, ferric ferrocyanide, guanine (natural pearl) mica, sericite, talc, magnesium carbonate, calcium carbonate, alumina hydroxide, barium sulfate, beta-carotene, annatto, chlorophyllin-copper complex, lycopene, henna, bronze powder, (metallic), copper powder (metallic), silver powder (metallic), gold powder (metallic), ultramarine blues and mixtures thereof.

40. The colored personal care composition of claim 37, wherein said pigment comprises an organic pigment.

41. The colored personal care composition of claim 40, wherein said organic pigment is selected from the group consisting of FD&C Blue#1, FD&C Yellow#5, FD&C Yellow#6, FD&C Yellow#7, FD&C Yellow#8, FD&C Yellow#10, FD&C Red#2, FD&C Red #4, FD&C Red#6, FD&C Red#7, FD&C Red #9, FD&C Red #17, FD&C Red #19, FD&C Red 21, FD&C Red #22, FD&C Red #27, FD&C Red #28, FD&C Red #30, FD&C Red #31, FD&C Red #33, FD&C Red 34, FD&C Red #36, FD&C Red #40, D&C Violet#2, Ext D&C Violet#2, FD&C Green#3, D&C Green#5, D&C Green#6, D&C Green#8, D&C Orange#4, D&C Orange #5, D&C Brown#1, D&C Black#2, carmine, and mixtures thereof.

42. The colored personal care composition of claim 32, wherein said pigment dispersion further comprises a rheology modifier.

43. The colored personal care composition of claim 42, wherein said rheology modifier comprises a fumed silica.

44. The colored personal care composition of claim 43, wherein the surface of said fumed silica is chemically modified.

45. The colored personal care composition of claim 44, wherein said modified fumed silica comprises silica dimethyl silylate.

46. A process for preparing a colored personal care composition comprising mixing a silicone pigment dispersion with at least one dermatological active agent.

47. The process of claim 46, wherein said silicone pigment dispersion exhibits a color deviation from a previously established color standard expressed by a delta-E value of 1.5 or less.

48. The process of claim 47, wherein said silicone polymer comprises a polysiloxane.

49. The process of claim 48, wherein said polysiloxane comprises a polyalkylsiloxane.

50. The process of claim 49, wherein said polyalkylsiloxane comprises the following chemical structure: 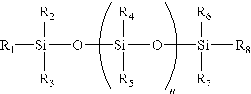
wherein R₁-R₇ are each independently selected from H, —OH, —CH₃, —CH₂CH₃, vinyl and combinations thereof; and
wherein n is an integer from about 3 to about 1,000,000.

51. The process of claim 50, wherein said polyalkylsiloxane comprises dimethylpolysiloxane.

52. The process of claim 51, wherein said pigment comprises an inorganic pigment, an organic pigment, or mixtures thereof.

53. The process of claim 52, wherein said pigment comprises an inorganic pigment.

54. The process of claim 53, wherein said inorganic pigment is selected from the group consisting of red iron oxide, yellow iron oxide, black iron oxide, brown iron oxide, titanium dioxide, zinc oxide, zirconium oxide, cerium oxide, chromium oxide, chromium hydroxide, chromium hydrate, manganese violet, ferric ferrocyanide, guanine (natural pearl) mica, sericite, talc, magnesium carbonate, calcium carbonate, alumina hydroxide, barium sulfate, beta-carotene, annatto, chlorophyllin-copper complex, lycopene, henna, bronze powder, (metallic), copper powder (metallic), silver powder (metallic), gold powder (metallic), ultramarine blues and mixtures thereof.

55. The process of claim 52, wherein said pigment comprises an organic pigment.

56. The process of claim 55, wherein said organic pigment is selected from the group consisting of FD&C Blue#1, FD&C Yellow#5, FD&C Yellow#6, FD&C Yellow#7, FD&C Yellow#8, FD&C Yellow#10, FD&C Red#2, FD&C Red #4, FD&C Red#6, FD&C Red#7, FD&C Red #9, FD&C Red #17, FD&C Red #19, FD&C Red 21, FD&C Red #22, FD&C Red #27, FD&C Red #28, FD&C Red #30, FD&C Red #31, FD&C Red #33, FD&C Red 34, FD&C Red #36, FD&C Red #40, D&C Violet#2, Ext D&C Violet#2, FD&C Green#3, D&C Green#5, D&C Green#6, D&C Green#8, D&C Orange#4, D&C Orange #5, D&C Brown#1, D&C Black#2, carmine, and mixtures thereof.

57. The process of claim 46, wherein said pigment dispersion further comprises a rheology modifier.

58. The process of claim 57, wherein said rheology modifier comprises a fumed silica.

59. The process of claim 58, wherein the surface of said fumed silica is chemically modified.

60. The process of claim 59, wherein said modified fumed silica comprises silica dimethyl silylate.

61. A process for providing a custom formulated pigment dispersion for preparing colored cosmetic or personal care composition comprising either (i) a cosmetically acceptable component or (ii) a dermatologically active agent and a silicone pigment dispersion comprising:
accepting a request from a party to prepare a custom formulated pigment dispersion to meet an established color standard having a desired color;
determining the type and amount of raw materials required to attain the desired color;
formulating the pigment dispersion with the type and amount of raw materials to attain to desired color; and
providing the custom formulated pigment dispersion to said party, wherein said pigment dispersion exhibits a color deviation expressed as a delta-E value of 1.5 or less from said established color standard.

62. The process of claim 61, wherein said pigment dispersion exhibits a color deviation from said established color standard reported by a delta-E value of 1.5 or less.
